(19) United States
(12) Patent Application Publication
(10) Pub. No.: US 2010/0241105 A1
(43) Pub. Date: Sep. 23, 2010

(54) SYSTEM FOR INTRODUCING IMPLANTS

(75) Inventors: Noah Meade, Atlanta, GA (US); Doug Evans, Snellville, GA (US)

Correspondence Address: Rutan & Tucker, LLP., 611 ANTON BLVD, SUITE 1400, COSTA MESA, CA, 92626, US

(73) Assignee: C.R. Bard, Inc., Murray Hill, NJ (US)

(21) Appl. No.: 11/993,089

(22) PCT Filed: Aug. 3, 2006
(86) PCT No.: PCT/US06/30369

§ 371 Date: Jun. 9, 2010

Related U.S. Application Data
(60) Provisional application No. 60/705,624, filed on Aug. 4, 2005.
(60) Provisional application No. 60/705,569, filed on Aug. 4, 2005.
(60) Provisional application No. 60/754,265, filed on Dec. 28, 2005.
(60) Provisional application No. 60/745,140, filed on Apr. 19, 2006.
(60) Provisional application No. 60/810,065, filed on Jun. 1, 2006.

(51) Int. Cl. A61B 17/00

(52) U.S. Cl. 606/1

(57) ABSTRACT

In one embodiment, an introducer system includes an introducer needle having a proximal end and a distal end and defining an inner lumen, the introducer needle further having an opening that provides access to the inner lumen, and a snare having an implant coupling element, the snare being configured to be positioned within the inner lumen of the introducer needle, wherein the snare is extendable from the introducer needle opening to an extended position in which the implant coupling element is positioned outside of the inner lumen and retractable to a retracted position in which the implant coupling element is positioned within the inner lumen.

### CROSS-REFERENCE TO RELATED APPLICATION

This application is related to copending U.S. provisional patent applications entitled “Introducer Needle with Extendable Implant Lasso”, filed on Dec. 28, 2005, and accorded Ser. No. 60/754,265, and “Introducer Needle With Implant Retrieval Snare”, filed on Apr. 19, 2006, and accorded Ser. No. 60/745,140, both of which are entirely incorporated herein by reference.

### BACKGROUND

Surgical devices referred to as “introducers” are often used to implant or “introduce” implantable devices within the body. For example, such introducers can be used to position within the pelvis mesh implants intended for treating urinary incontinence or performing prolapse repair.

Positioning an implant within the human body, such as within the pelvis, can be challenging due to the anatomy of the body and the placement of the implant that may be required to treat a given ailment. For instance, the treatment of rectocele, a condition in which the rectum encroaches on the vagina, may require accessing the vaginal vault from a position deep within the pelvis so as to form a passage in which a portion, such as an anchoring arm, of the implant can be placed. Formation of such a passage typically requires a relatively high degree of skill.

Further complicating implantation of a rectocele implant, or other such pelvic implant, is the need to draw the implant into the body and through the formed passage. In present techniques, a needle is passed through a pelvic incision, through the soft tissue of the pelvis, into the vagina, down through the vagina, and out the vaginal introitus to enable the implant to be connected to the needle so that the needle may then be withdrawn with the implant in tow to position the implant within the formed passage. Given the configuration and dimensions of the human pelvis and its organs, it can be difficult to navigate a needle through such a tortuous path without causing damage to or otherwise disrupting the tissues of the pelvis, such as the pelvic floor muscles.

### BRIEF DESCRIPTION OF THE DRAWINGS

The disclosed systems can be better understood with reference to the following drawings. The components in the drawings are not necessarily to scale.

FIG. 1 illustrates a first embodiment of an introducer system.

FIG. 2 is a perspective view of an introducer shown in FIG. 1.

FIG. 3 illustrates passage of a snare shown in FIG. 1 through the introducer shown in FIG. 2.

FIG. 4 illustrates a second embodiment of an introducer system.

FIG. 5 is a perspective view of an introducer shown in FIG. 4.

FIG. 6 illustrates passage of a snare shown in FIG. 1 through the introducer shown in FIG. 5.

FIGS. 7A-7K illustrate steps performed in a first embodiment of a method for implanting a pelvic implant within the body.

FIGS. 8A and 8B illustrate steps performed in a second embodiment of a method for implanting a pelvic implant within the body.

FIG. 9 is a side view of an alternative embodiment of a snare that can be used in an introducer system.

FIG. 10 is a partial front view of the snare of FIG. 9, illustrating an implant coupling element of the introducer.

FIG. 11 is a perspective view of an alternative embodiment of an introducer that can be used in an introducer system.

FIG. 12 illustrates securing of a snare to the introducer shown in FIG. 11 using a cleat of the snare.

### DETAILED DESCRIPTION

As described above, it can be difficult to position an implant within the body. That may particularly be the case in relation to positioning a pelvic implant intended for use in treating incontinence or performing prolapse repair. For example, as described above, a surgeon may need to access a point deep within the pelvis, such as the vaginal vault, with an introducer and connect an implant to the introducer at a point outside of the body to enable the implant to be drawn through a passage formed in the soft tissues of the pelvis by the introducer. It is difficult to perform such a procedure with current introducers given that the introducer must traverse a tortuous path to extend outside of the body from a point deep within the pelvis.

Disclosed herein are introducer systems that simplify implantation of an implantable device, such as a pelvic implant. In some embodiments, the introducer system comprises a snare that can be extended from a tip of an introducer needle to a position outside of the body when the tip is positioned at a point within the body, such as within vagina. In such a case, an implant can be coupled to the extended snare and the snare can then be retracted to pull the implant through the body and at least to the tip of the introducer needle. In some embodiments, both the snare and the implant can further be drawn through the introducer needle such that the implant traverses the passage formed by the introducer needle without direct contact with the tissues of the passage, thereby reducing irritation to the soft tissues in which the passage is formed.

In the following, various embodiments of systems and methods are described in detail. Although specific embodiments are presented, those embodiments are mere exemplary implementations of the disclosed systems and methods and it is noted that other embodiments are possible. All such embodiments are intended to fall within the scope of this disclosure.

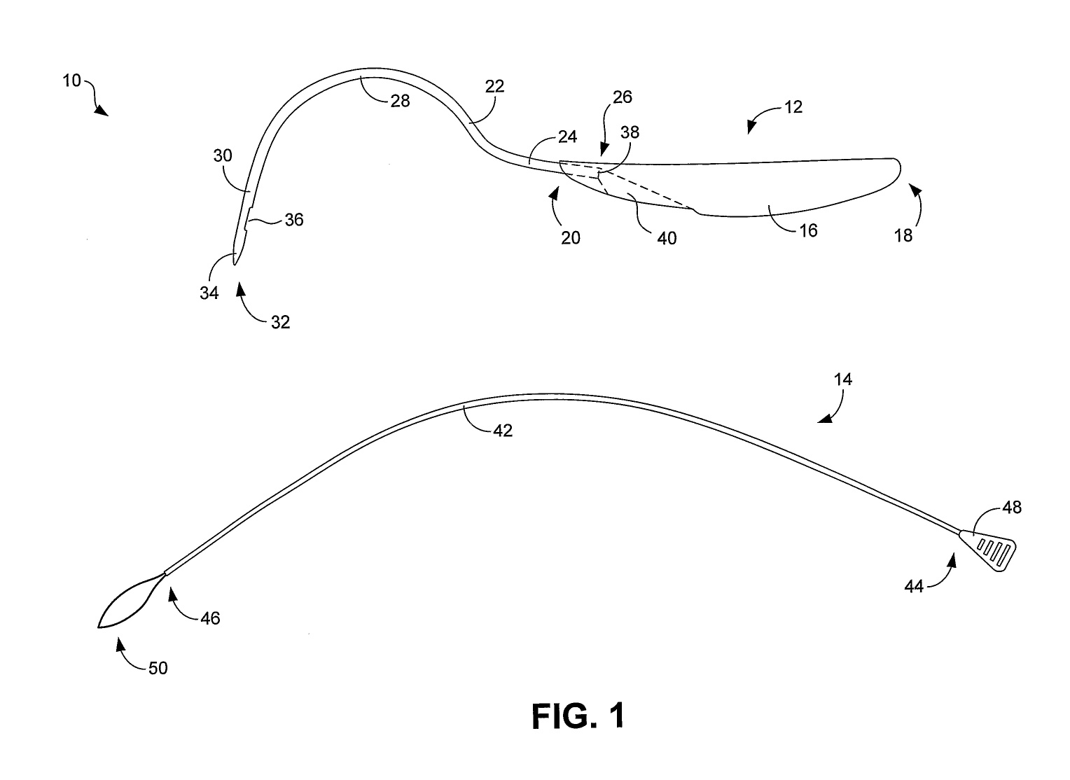

FIG. 1 illustrates a first embodiment of an introducer system 10. The introducer system 10 is well suited for use in performing prolapse repair, such as anterior prolapse repair and treating cystocele. As indicated in FIG. 1, the system 10 includes an introducer 12 and a snare 14. The introducer 12 comprises a handle 16 that includes a proximal end 18 and a distal end 20. The handle 16 is generally sized and shaped to fit within a surgeon's hand and, as depicted in FIG. 1, can be curved to facilitate firm gripping.

A needle 22 extends from the distal end 20 of the handle 16. As shown in FIG. 1, at least a portion of the needle 22 is curved. In the embodiment of FIG. 1, the needle 22 comprises a first generally straight portion 24 adjacent its proximal end 26, a curved portion 28 in a central region, and a second generally straight portion 30 adjacent its distal end 32. Formed at the distal end 32 is a blunt point or tip 34 that is configured to dissect soft tissue as the needle 22 is passed through the body.

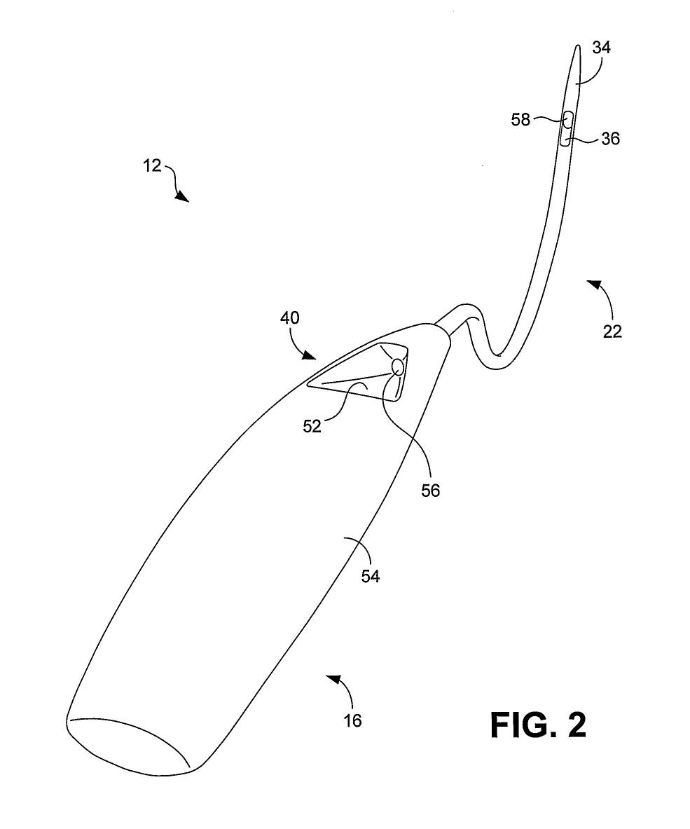

The needle 22 is hollow so as to form a cannula through which the snare 14 can be passed. More particularly, the needle 22 forms an inner lumen that extends from a first opening 36 of the needle to a second opening 38 of the needle. In the embodiment shown in FIG. 1, the first opening 36 is positioned adjacent the distal end 32 and the second opening 38 is positioned adjacent the proximal end 26. The second opening 38 is in open communication with a port 40 that is formed in the handle 16. As is described in greater detail below, the snare 14 can be passed through the port 40 and the second opening 38 to position the snare within the needle 22. The configuration of the port 40 is described in relation to FIG. 2.

In terms of materials, the handle 16 can be constructed of any suitable rigid material, such as a metal or a polymeric material. The needle 22 can be constructed of a biocompatible, strong material, such as stainless steel. In some embodiments, the handle 16 and needle 22 can be composed of the same material and may even be unitarily formed together so as to have a monolithic configuration.

With continued reference to FIG. 1, the snare 14 comprises an elongated shaft 42 having a proximal end 44 and a distal end 46. The shaft 42 is flexible so as to enable the shaft to easily adapt to the contours of the needle inner lumen and any body passages along which the snare is to travel. In some embodiments, the shaft 42 comprises a hollow tube through which a wire passes. In such cases, the shaft 42 can be constructed of a suitable flexible biocompatible material, such as a polymeric material. In other embodiments, the shaft 42 is solid and can be made of a polymeric material or a metal material, such as stainless steel or nitinol.

Provided at the proximal end 44 of the snare 14 is a grip element 48 that, as described below, is used to manipulate the snare relative to the introducer 12. Provided at the distal end 46 of the snare 14 is an implant coupling element 50 that is configured to couple to and secure an implant that is to be positioned with the body. In the illustrated embodiment, the coupling element 50 is formed as a loop. Such a loop can be formed from a flexible wire constructed of a polymeric or metal material. In such a case, the wire can extend from the gripping element 48, through the shaft 42, and terminate in a loop. In some embodiments, nitinol is suitable for the construction of the coupling element 50 due to nitinol's shape memory characteristics. In particular, when nitinol is used, the coupling element 50 can easily be compressed to pass through the needle inner lumen, but can readily spring back to its original shape (e.g., loop shape) after emerging from the needle 22. In some embodiments, the shaft 42 and the coupling element 50 comprise a unitarily-formed element, such as an elongated wire that extends from the gripping element 48 and terminates in a loop. In such cases, the shaft 42 need not comprise a tube.

FIG. 2 is a perspective view of the introducer 12. As indicated in that figure, the port 40 of the handle 16 is formed by one or more surfaces 52 that extend inwardly from an outer surface 54 of the handle to an orifice 56 that is aligned with the second opening 38 of the needle 22 (FIG. 1). As is also visible in FIG. 2, the needle 22 includes a snare-deflecting surface 58 positioned within the first opening 36 that urges the snare 14 (FIG. 1) out from the needle when the snare is pushed against the surface.

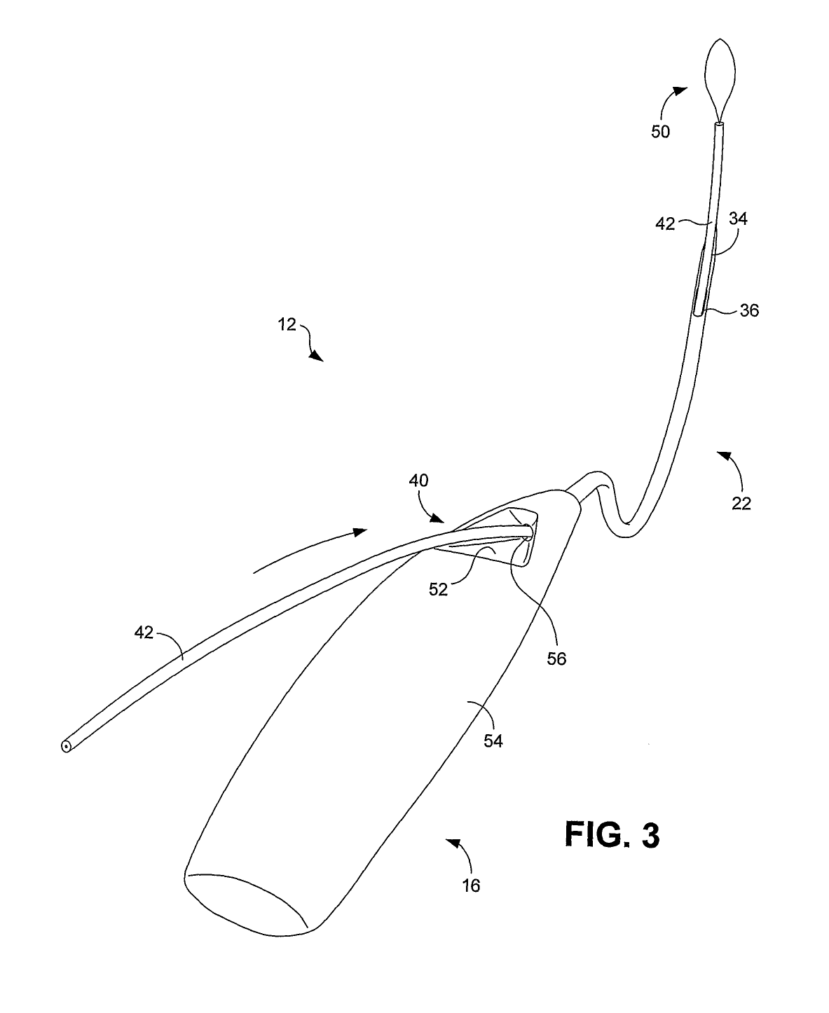

With the above-described system configuration, the snare 14 can be inserted through the port 40 and orifice 56 of the introducer handle 16, moved into the inner lumen of the introducer needle 22, pushed through the needle inner lumen, and made to exit the needle through the first opening 36. The result of that process is illustrated in FIG. 3.

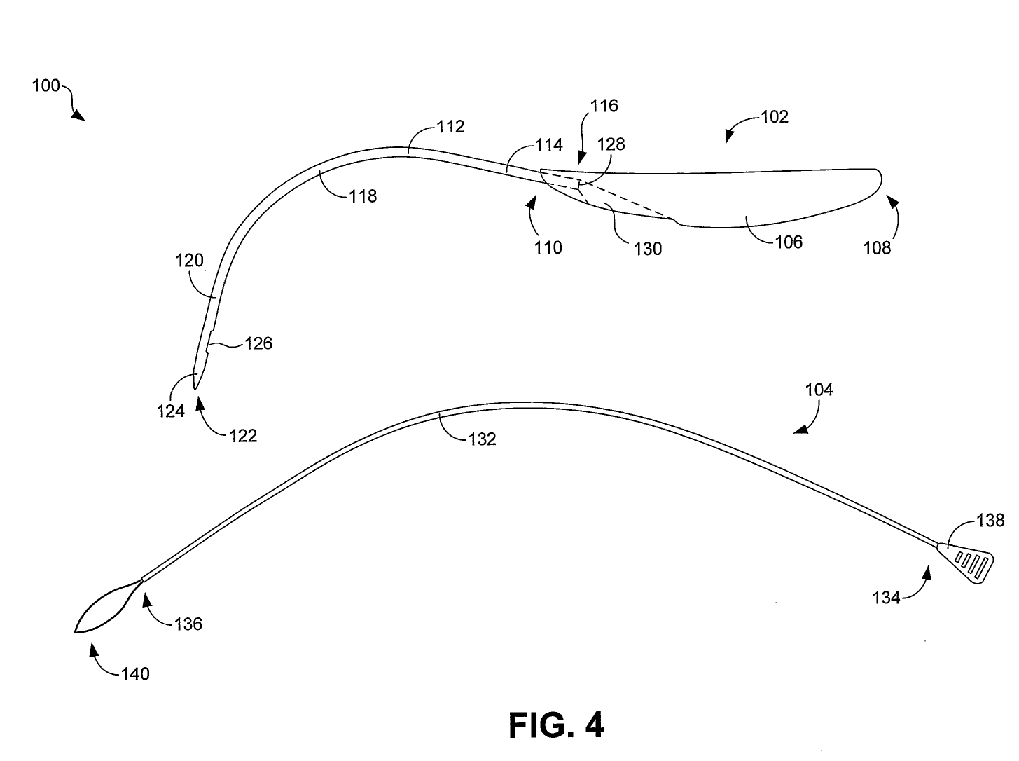

FIG. 4 illustrates a second embodiment of an introducer system 100. The introducer system 100 is similar to the system 10 described in relation to FIGS. 1-3, although the system 100 is configured for use in performing posterior prolapse repair and treating rectocele. As indicated in FIG. 4, the system 100 includes an introducer 102 and a snare 104. The introducer 102 comprises a handle 106 that includes a proximal end 108 and a distal end 110. The handle 106 is generally sized and shaped to fit within a surgeon's hand and, as depicted in FIG. 4, can be curved to facilitate firm gripping.

A needle 112 extends from the distal end 110 of the handle 102. As shown in FIG. 4, at least a substantial portion of the needle 112, like needle 22 (FIG. 1) is curved. In the embodiment of FIG. 4, however, the needle 112 is longer and straighter to enable passage of the needle deep into the pelvis. The needle 112 comprises a first generally straight portion 114 adjacent its proximal end 116, a curved portion 118 in a central region, and a second generally straight portion 120 adjacent its distal end 122. Formed at the distal end 122 is a blunt point or tip 124 that is configured to dissect soft tissue as the needle 112 is passed through the body.

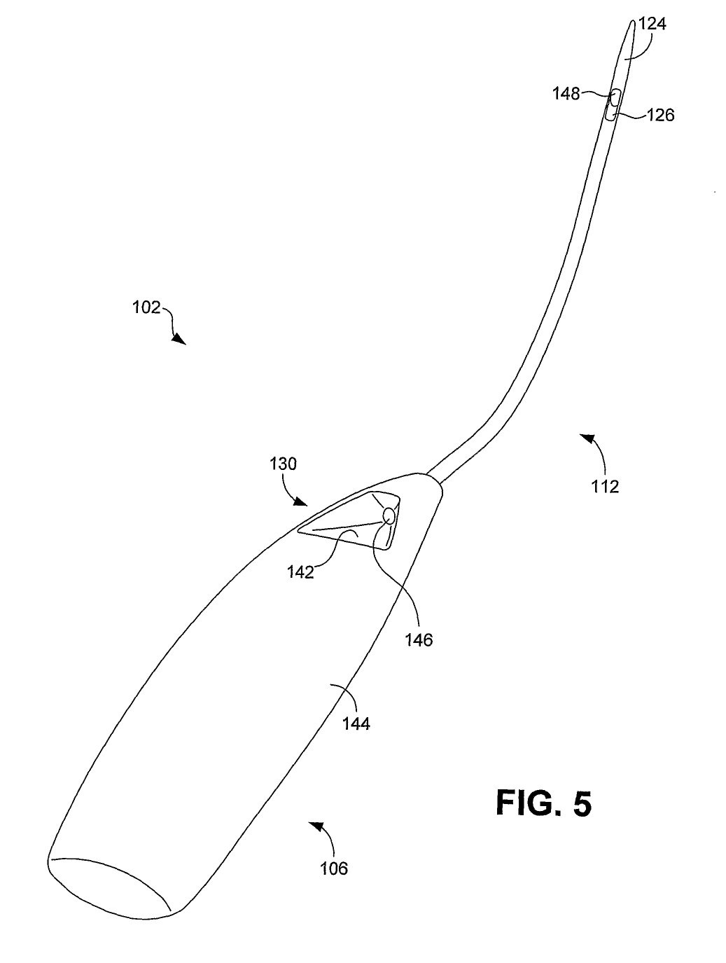

The needle 112 is hollow so as to form a cannula through which the snare 104 can be passed. More particularly, the needle 112 forms an inner lumen that extends from a first opening 126 of the needle to a second opening 128 of the needle. In the embodiment shown in FIG. 4, the first opening 126 is positioned adjacent the distal end 122 and the second opening 128 is positioned adjacent the proximal end 116. The second opening 128 is in open communication with a port 130 that is formed in the handle 106. As is described in greater detail below, the snare 104 can be passed through the port 130 and the second opening 128 to position the snare within the needle 112. The configuration of the port 130 is described in relation to FIG. 5.

In terms of materials, the handle 106 can be constructed of any suitable rigid material, such as a metal or a polymeric material. The needle 112 can be constructed of a biocompatible, strong material, such as stainless steel. In some embodiments, the handle 106 and needle 112 can be composed of the same material and may even be unitarily formed together so as to have a monolithic configuration.

With continued reference to FIG. 4, the snare 104 comprises an elongated shaft 132 having a proximal end 134 and a distal end 136. The shaft 132 is flexible so as to enable the shaft to easily adapt to the contours of the needle inner lumen and any body passages along which the snare is to travel. In some embodiments, the shaft 132 comprises a hollow tube through which a wire passes. In such cases, the shaft 132 can be constructed of a suitable flexible biocompatible material, such as a polymeric material. In other embodiments, the shaft 132 is solid and can be made of a polymeric material or a metal material, such as stainless steel or nitinol.

Provided at the proximal end 134 of the snare 104 is a grip element 138 that, as described below, is used to manipulate the snare relative to the introducer 102. Provided at the distal end 136 of the snare 104 is an implant coupling element 140 that is configured to couple to and secure an implant that is to be positioned with the body. In the illustrated embodiment, the coupling element 140 is formed as a loop. Such a loop can be formed from a flexible filament, such as a wire, constructed of a polymeric or metal material. In such a case, the wire can extend from the gripping element 138, through the shaft 132, and terminate in a loop. In some embodiments, nitinol is suitable for the construction of the coupling element 140 due to nitinol's shape memory characteristics. In particular, when nitinol is used, the coupling element 140 can easily be compressed to pass through the needle inner lumen, but can readily spring back to its original shape (e.g., loop shape) after emerging from the needle 112. In some embodiments, the shaft 132 and the coupling element 140 comprise a unitarily-formed element, such as an elongated wire that extends from the gripping element 138 and terminates in a loop. In such cases, the shaft 132 need not comprise a tube.

FIG. 5 is a perspective view of the introducer 102. As indicated in that figure, the port 130 of the handle 106 is formed by one or more surfaces 142 that extend inwardly from an outer surface 144 of the handle to an orifice 146 that is aligned with the second opening 128 of the needle 112 (FIG. 4). As is also visible in FIG. 5, the needle 112 includes a snare-deflecting surface 148 positioned within the first opening 126 that urges the snare 14 (FIG. 4) out from the needle when the snare is pushed against the surface.

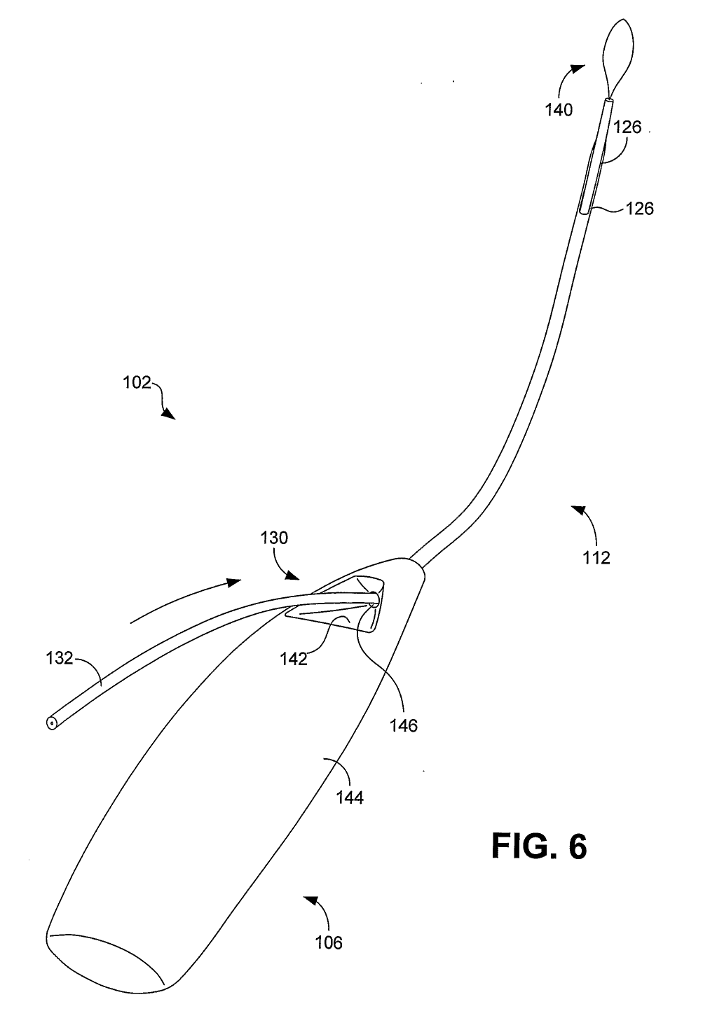

With the above-described system configuration, the snare 104 can be inserted through the port 130 and orifice 146 of the introducer handle 106, moved into the inner lumen of the introducer needle 112, pushed through the needle inner lumen, and made to exit the needle through the first opening 126. The result of that process is illustrated in FIG. 6.

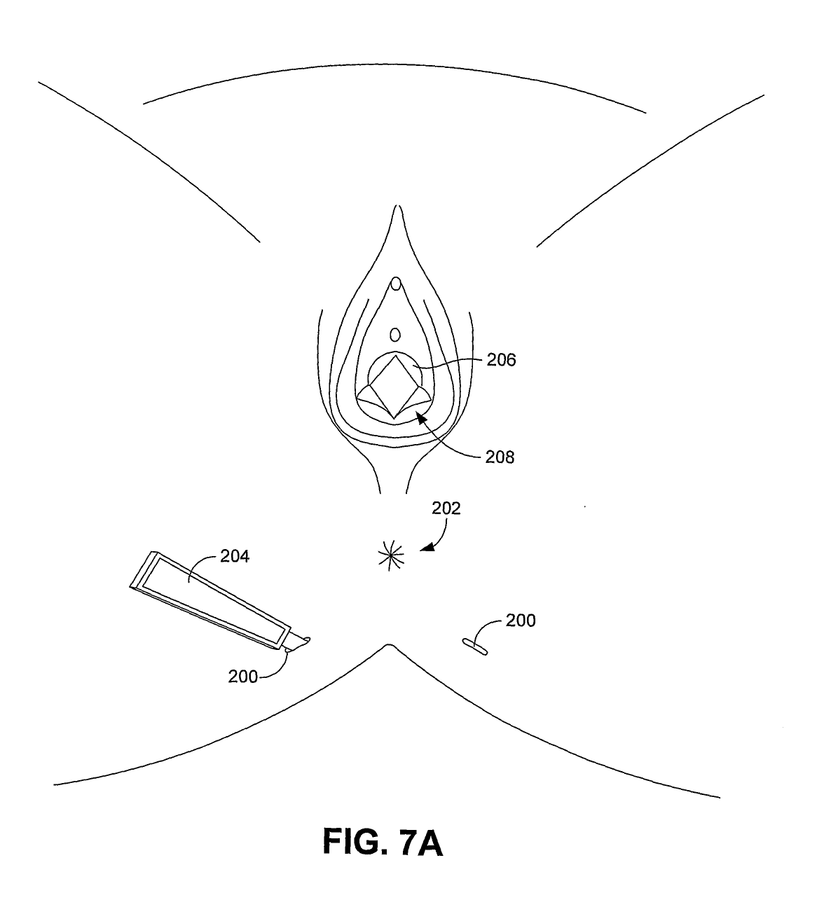

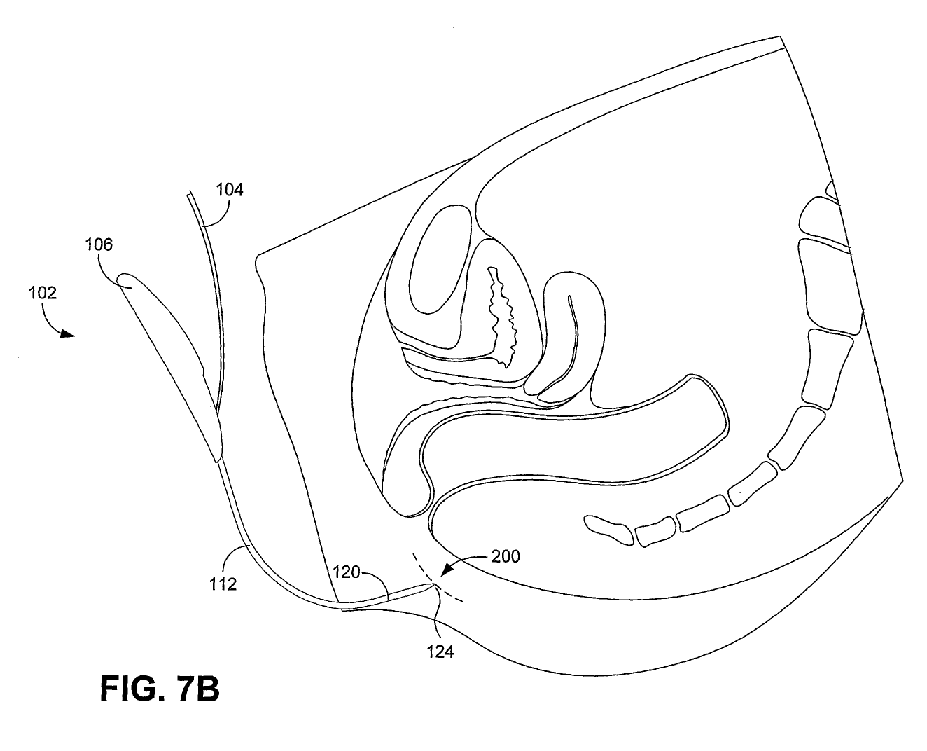

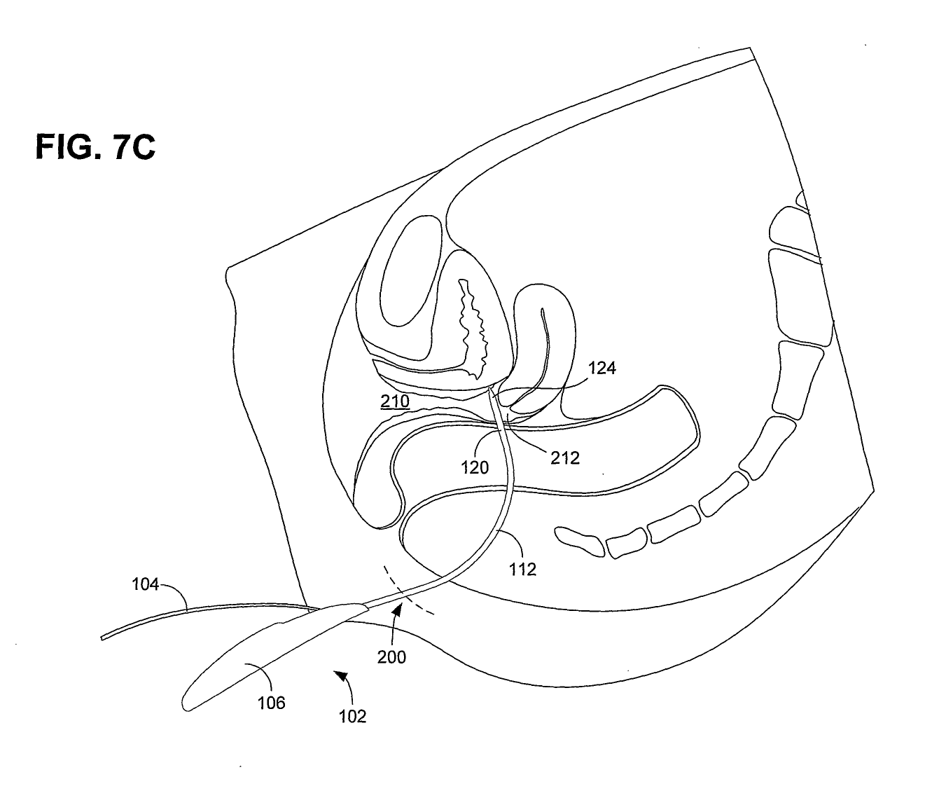

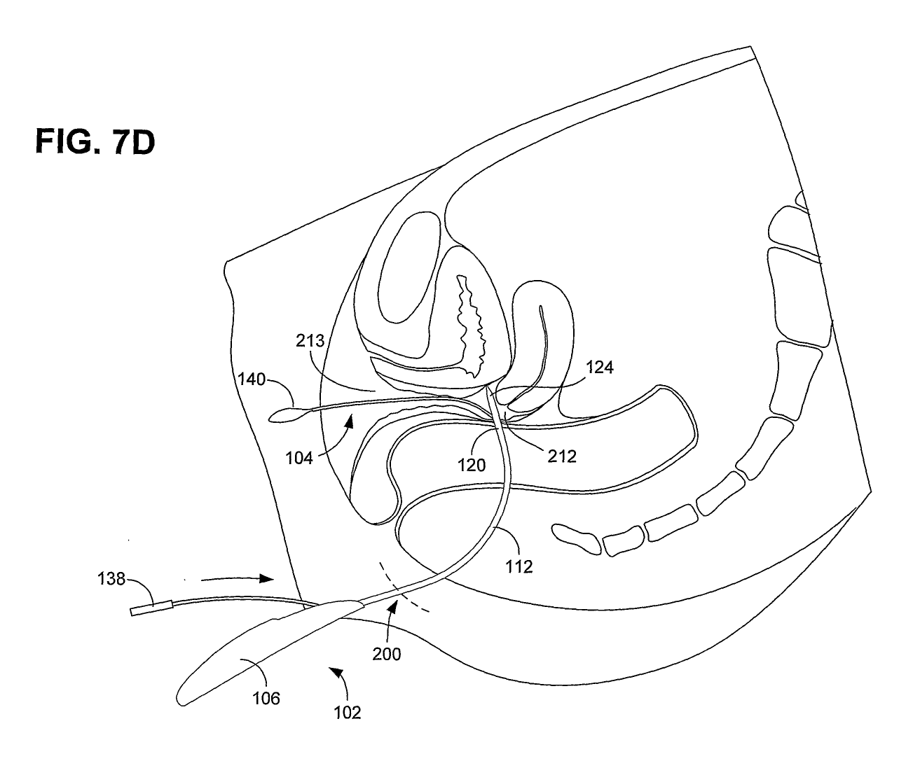

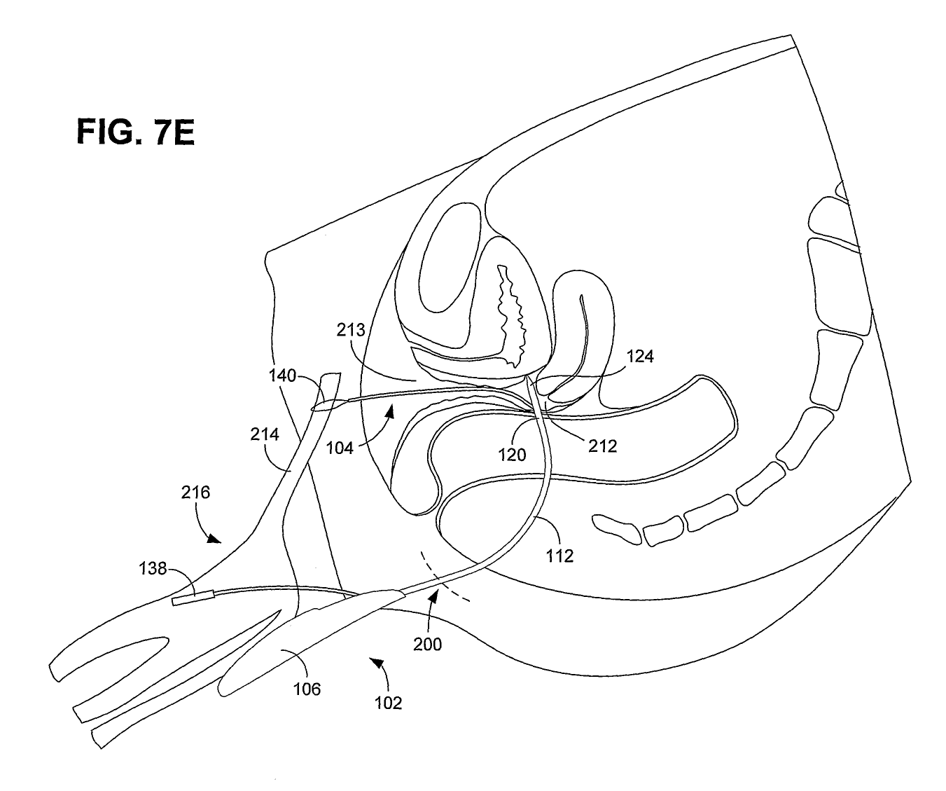

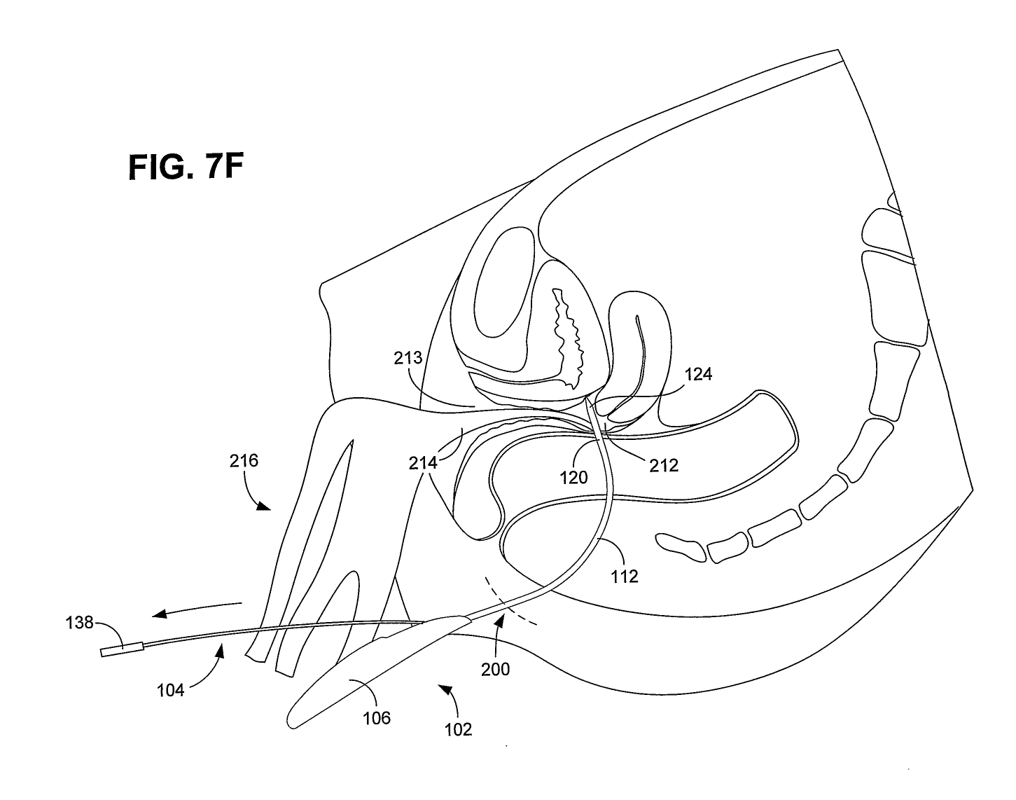

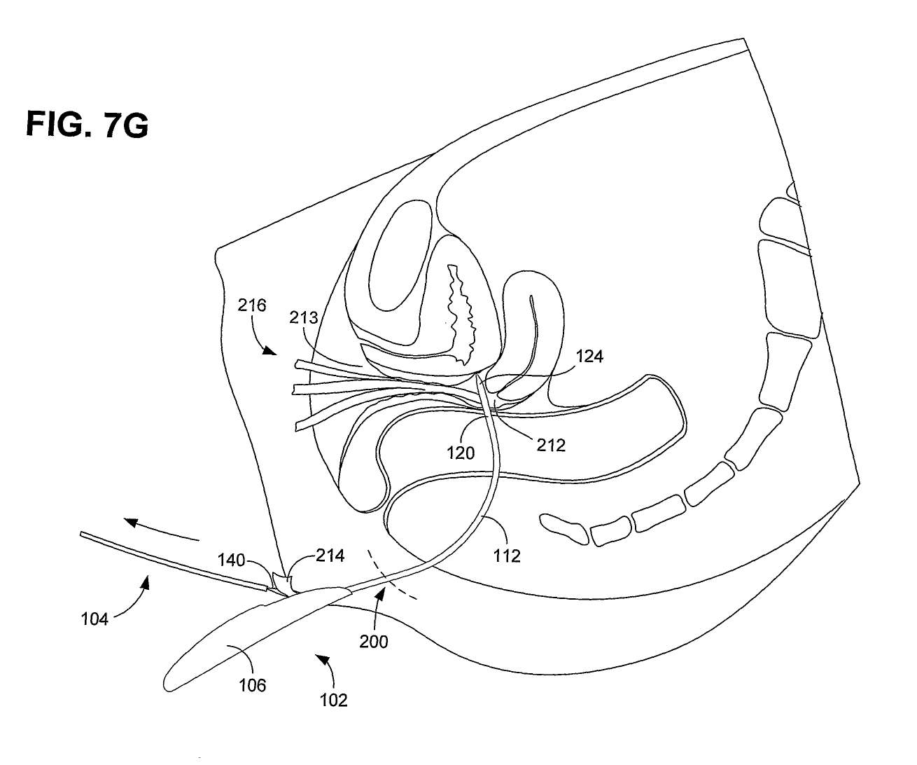

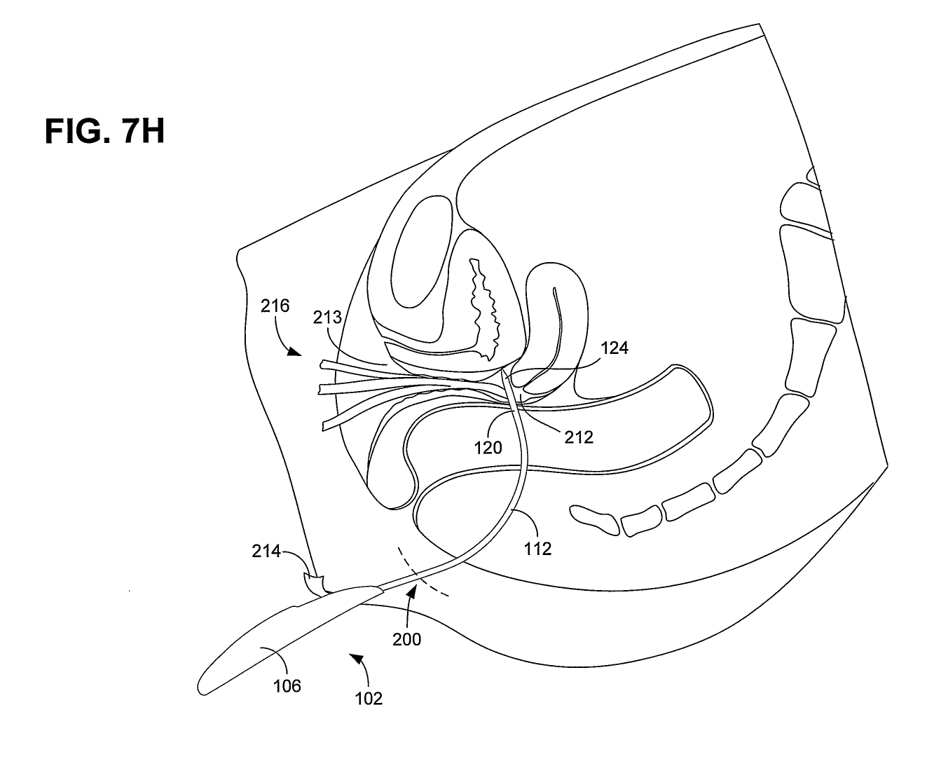

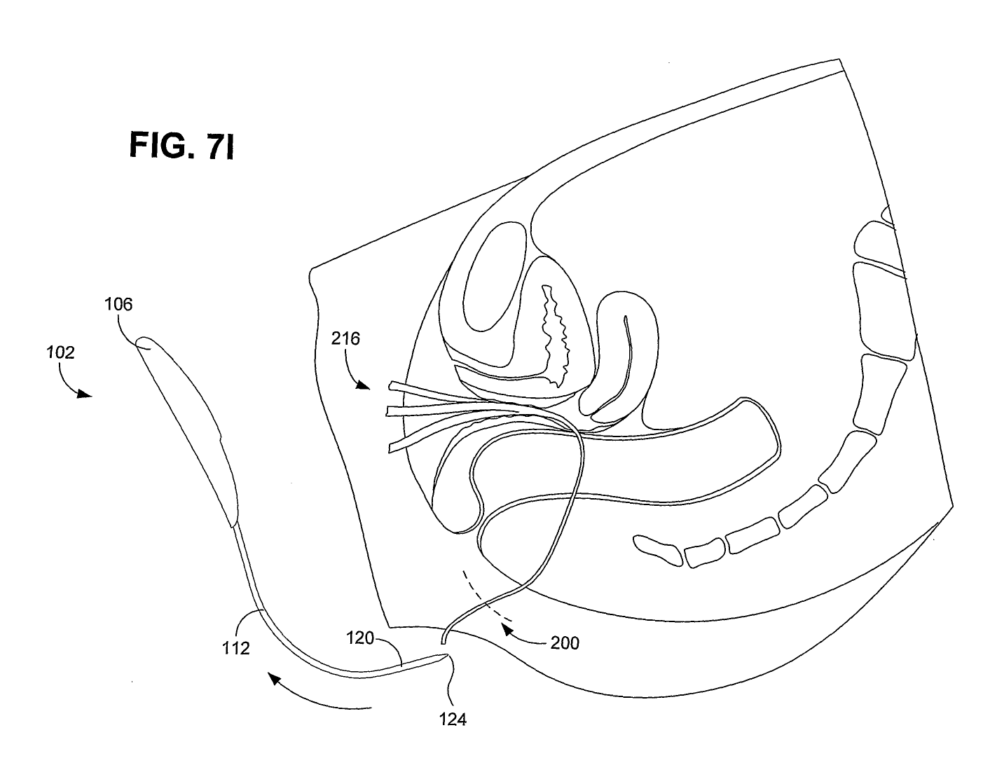

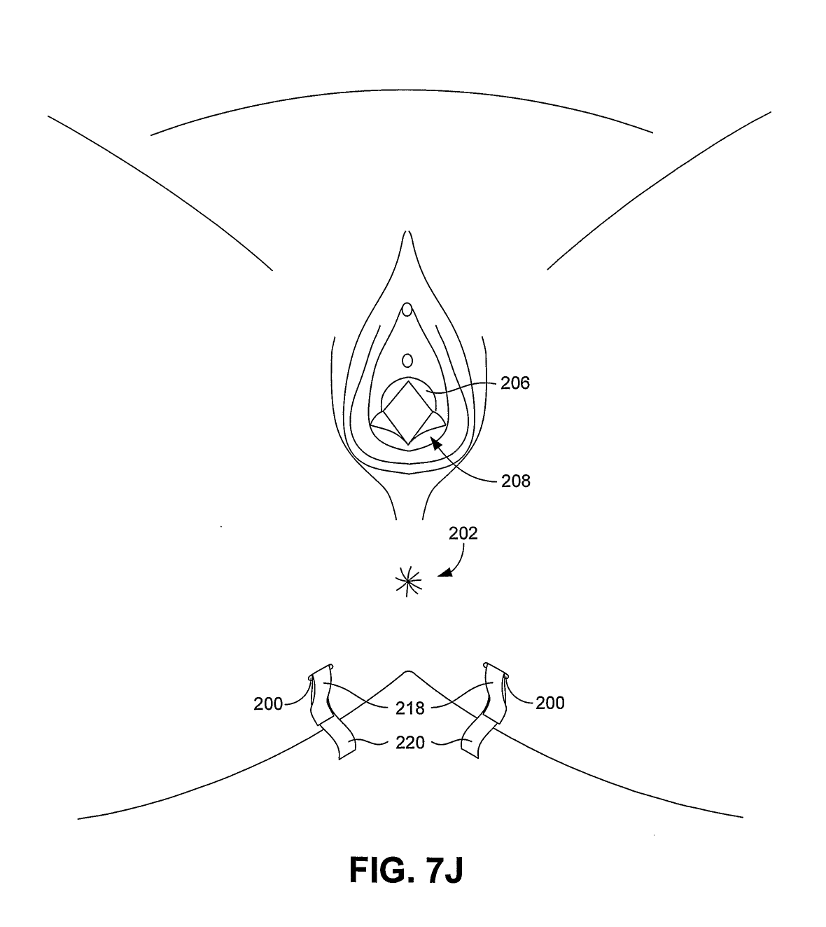

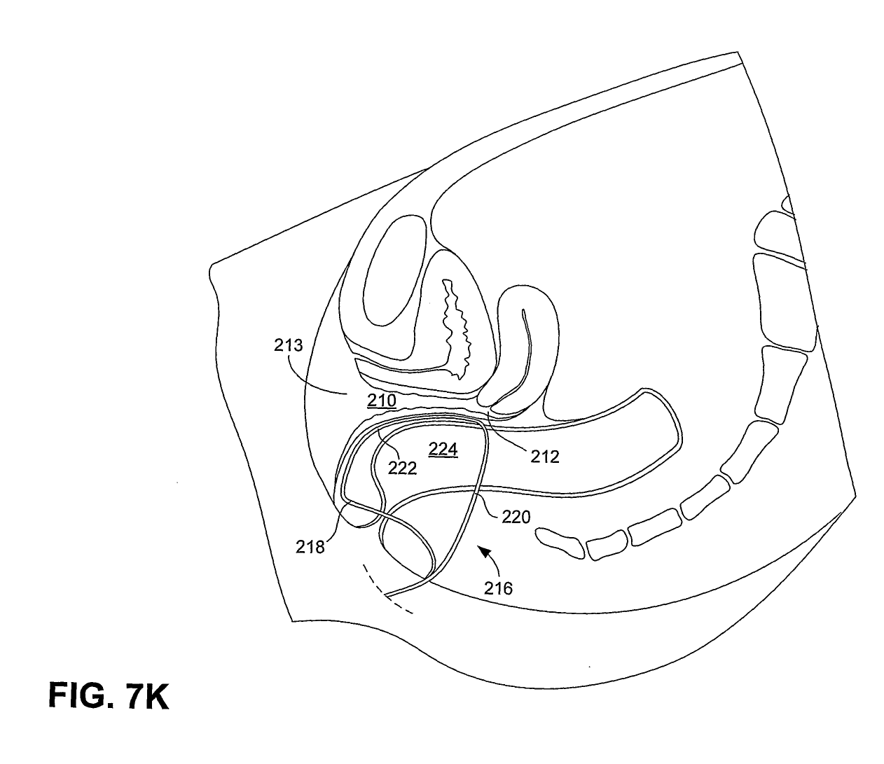

FIGS. 7A-7K illustrate a process for implanting an article using a system of the disclosure. More particularly, FIGS. 7A-7K illustrate a procedure for implanting a posterior prolapse repair implant between the vagina and the rectum using the introducer system 100 shown in FIG. 4. Although a posterior repair procedure is depicted in FIGS. 7A-7K and is described in detail in the following for purposes of describing the manner in which the disclosed introducer systems can be used to introduce an implant, it is to be understood that the procedure is described for purposes of example only. As stated above, similar systems may be used to implant other implants in other surgical procedures, such as anterior prolapse repair or treatment of urinary incontinence.

Beginning with FIG. 7A, small pararectal incisions 200 are made on either side of the anus 202 with a sharp device, such as a scalpel 204. By way of example, the incisions 200 are made 2-3 centimeters (cm) posterior and lateral to the anus 202. In addition, a midline incision is made in the posterior vaginal wall 206 to form an opening 208 that extends from the vaginal introitus to the vaginal apex to provide access to the space between the vagina and the rectum. The vaginal mucosa may then be dissected away from the rectum using blunt and/or sharp dissection.

Turning to FIG. 7B, the tip 124 of the introducer needle 112 is positioned at one of the incisions 200 with the introducer 102 oriented so that the handle 106 is substantially vertical and the second straight portion 120 of the needle is substantially parallel to the vagina 210. Referring next to FIG. 7C, the introducer needle 112 is passed through the incision 200 and through the soft tissue of the pelvis toward the ischial spine (not shown). As the needle 112 passes through the soft tissue, the introducer 102 is rotated so that the second straight portion 120 approaches a vertical orientation, as indicated in the figure. The needle tip 124 is advanced through the posterior vaginal wall and into the vaginal vault 212 such that the tip is positioned within the vagina. That process can be aided by placing a finger within the vagina to guide the needle tip 124 into position.

With reference to FIG. 7D, the snare 104, which can have been positioned already within the introducer 102 or later inserted therein, is extended from a retracted position in which the implant coupling element 140 is contained within the inner lumen of introducer needle 112 to an extended position in which the coupling element extends beyond the first opening 126 (FIG. 4) of the needle. The snare 104 is then extended through the introducer 102, for example using the gripping element 138, until the implant coupling element 140 passes out from the vaginal introitus 213, as indicated in FIG. 7D.

Referring next to FIG. 7E, a relatively long anchoring arm 214 of an implant 216 is coupled to the implant coupling element 140. By way of example, the implant 216 comprises a flexible mesh implant such that the arm 214 can be simply passed through the loop of the coupling element to secure the implant to the snare 104.

Turning to FIG. 7F, the snare 104 is retracted back into the introducer needle 112, for example using the gripping element 138, such that the implant coupling element 140 is again contained within the inner lumen of the needle. Due to the coupling between the implant 216 and the snare 104, a portion of the anchoring arm 214 may also be contained within the needle inner lumen. In some embodiments, a stop mechanism (not shown) can be provided within the needle inner lumen so as to limit the extent to which the snare 104 can be retracted into the needle inner lumen. For example, a stop (not shown), such as a bulbous portion, can be provided along the snare 104 adjacent the implant coupling element 140 that will abut a mating surface within the needle inner lumen, such as a constriction, adjacent the needle tip 124 so that the implant coupling element can be drawn into the needle inner lumen, but not farther through the needle inner lumen. Such a stop mechanism facilitates simultaneous withdrawal of the snare 104 and the needle 112. In other embodiments, the snare 104 need not be retracted back into the introducer needle 112 at all. In such an embodiment, the needle 112 and snare 104 can be withdrawn from the patient together with the snare in the extended position, if desired.

With reference next to FIG. 7G, at least a portion of the implant arm 214 can be pulled through the inner lumen of the introducer needle 112 so as to position the anchoring arm 214 in the passage that extends between the incision 200 and the vagina 210, which was formed by the needle. Notably, because the implant arm 214 is placed into that position while still contained within the needle 112, damage to the soft tissues in which the passage has been formed is reduced, as is the friction that resists such positioning. As shown in FIG. 7G, the snare 104 can be retracted to the point at which the implant coupling element 140 and the anchoring arm 214 exit the introducer handle 106. At that point, the anchoring arm 214 has been properly positioned within the body for subsequent adjustment, if necessary. As mentioned above, however, the snare 104 can, alternatively, be retracted to a limited extent due to the provision of a stop mechanism, or can not be retracted at all, as desired by the surgeon performing the procedure.

Assuming the snare 104 is retracted to the point at which it exits the introducer handle, the anchoring arm 214 is released from the implant coupling element 140, as indicated in FIG. 7H. Then, as indicated in FIG. 7I, the introducer needle 112 can be withdrawn from the body through the incision 200, thereby leaving the anchoring arm 214 in place within the tissues of the pelvis with a portion of the arm extending out from the incision. As mentioned above, the snare 104 can be withdrawn from the body simultaneous to withdrawal of the needle 112 in cases in which the snare is not withdrawn from the needle inner lumen (e.g., due to provision of a stop mechanism) or in cases in which the snare is not retracted back into the needle inner lumen after extension at all. The same result is achievable in such cases, however, given that the anchoring arm 214 can still be drawn through the passage formed by the needle 112 until a portion of the arm extends from the incision 200. The primary difference in such cases is that the anchoring arm 214 is in direct contact with the soft tissue of the passage as it passes through the passage instead of travelling through the needle inner lumen.

At this point, a similar procedure can be followed for positioning the opposite arm of the implant 216 using the other pararectal incision 200. That is, the introducer needle 112 can be passed through the incision 200 to the vaginal vault 212 on the opposite side of the vagina 210 and the opposite implant arm can be positioned in the passage formed by the needle. In addition, the relatively short arms of the implant can be positioned in other passages extending from the incisions on opposite sides of the vagina 210 to a position adjacent the vaginal introitus 213. Once that has been completed, a portion of a relatively short arm 218 and a portion of a relatively long arm 220 extends out from each pararectal incision 200, as indicated in FIG. 7J, and a central body 222 (FIG. 7K) of the implant 216 can be positioned between the vagina 210 and the rectum 224 to provide a support structure that prevents encroachment of the rectum into the vaginal space. Finally, the implant arms 218, 220 can be appropriately tensioned, for example by pulling excess length out from the incisions 200, and the portions of the arms that extend outside of the body trimmed. The final result of the implantation is illustrated in FIG. 7K, with the implant body 222 positioned between the vagina 210 and the rectum 224.

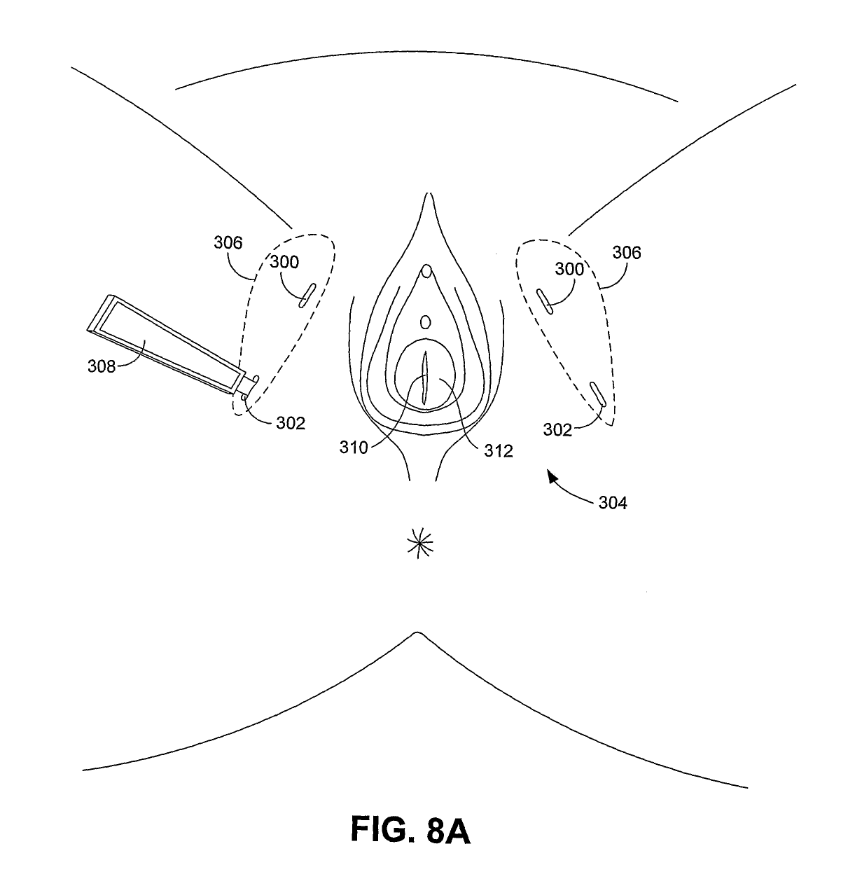

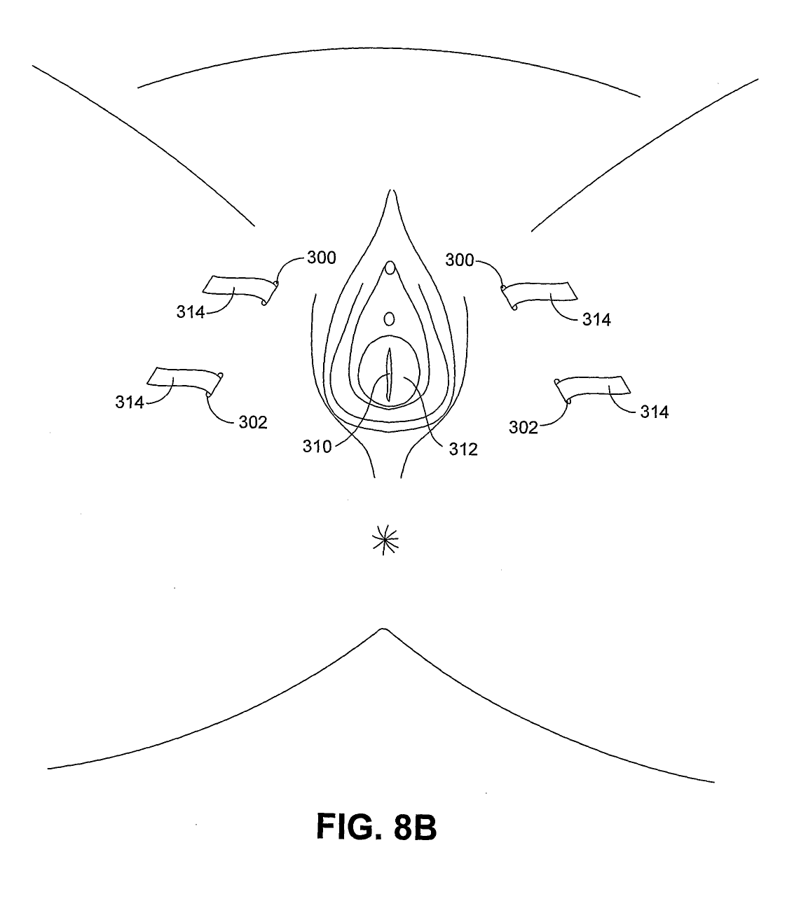

As described above, other implantation procedures can be performed using similar introducer systems. For example, anterior prolapse repair can be performed. To perform such a procedure, similar steps to those described above are completed. The primary differences include the shape of the implant, the location of the incisions made in the pelvis, and the positioning of the implant within the pelvis. As shown in FIG. 8A superior and inferior incisions 300 and 302 can be made in the paravaginal region 304 in alignment with the obturator foramina 306 of the pubic bone. Again, those incisions 300 and 302 can be made with a sharp device, such as a scalpel 308. In addition, a midline incision 310 can be made in the anterior vaginal wall 312 to provide access to the space between the vagina and the urethra. Each of four arms can be positioned within passages that extend from the incisions 300 and 302 to the vagina to position a body of the implant between the vagina and the urethra. As shown in FIG. 8B, portions 314 of the arms extend from the incisions 300 and 302 can then be trimmed as described above in relation to the posterior prolapse repair procedure.

As is also described above, the introducer systems can be used to treat urinary incontinence. In such a procedure, similar steps are performed except that the implant can comprise a urethral sling that is positioned below the urethra to provide support to the urethra. The ends of the sling can, for example, be passed through and/or embedded in the obturator foramina, or can be otherwise secured to hard or soft tissue of the pelvis.

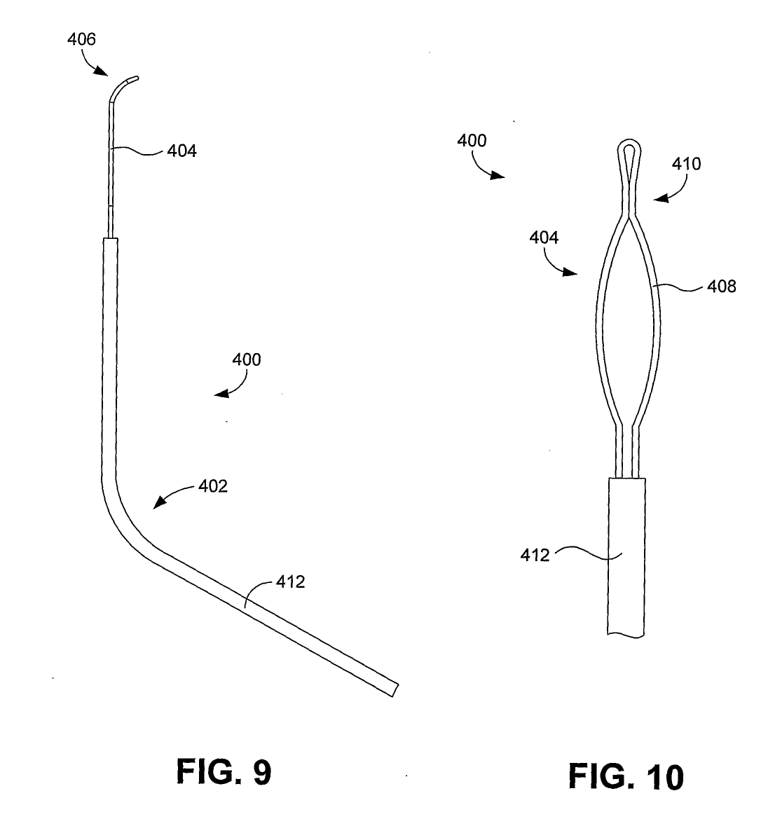

FIGS. 9 and 10 illustrate an alternative embodiment of a snare 400. Referring first to FIG. 9, the snare 400 can be formed as a wire constructed of a suitable metal material, such as stainless steel or nitinol. The snare 400 is pre-shaped to have a bend 402 that facilitates manipulation of the snare when positioned within the vagina or other body passage in which it is used. In particular, the bend 402 provides steering capability to the snare 400 so that the implant coupling element 404 of the snare can be moved in a desired direction, for example by twisting the snare using a grip element of the snare (not shown). As indicated in FIG. 9, the implant coupling element 404 comprises a further bend 406 that reduces the likelihood of snagging of the snare 400 within the vagina once the snare has been extended from its introducer needle.

Turning to FIG. 10, the implant coupling element 404 comprises a loop 408 and a constriction 410 that is, for example, positioned at a distal end of the loop. With such a configuration, an implant can be securely held by the implant coupling element 404 by first passing a portion of the implant through the loop 408 and then passing the implant portion into the constriction 410, such that the implant is securely clamped by the constriction. As is apparent from FIG. 10, the implant coupling element 404 can be formed from a wire that extends from a shaft 412 and forms the loop 408 and the constriction 410. In an alternative arrangement, the snare 400 can only comprise one or more wires that form the loop 408 and constriction 410, as well as the shaft.

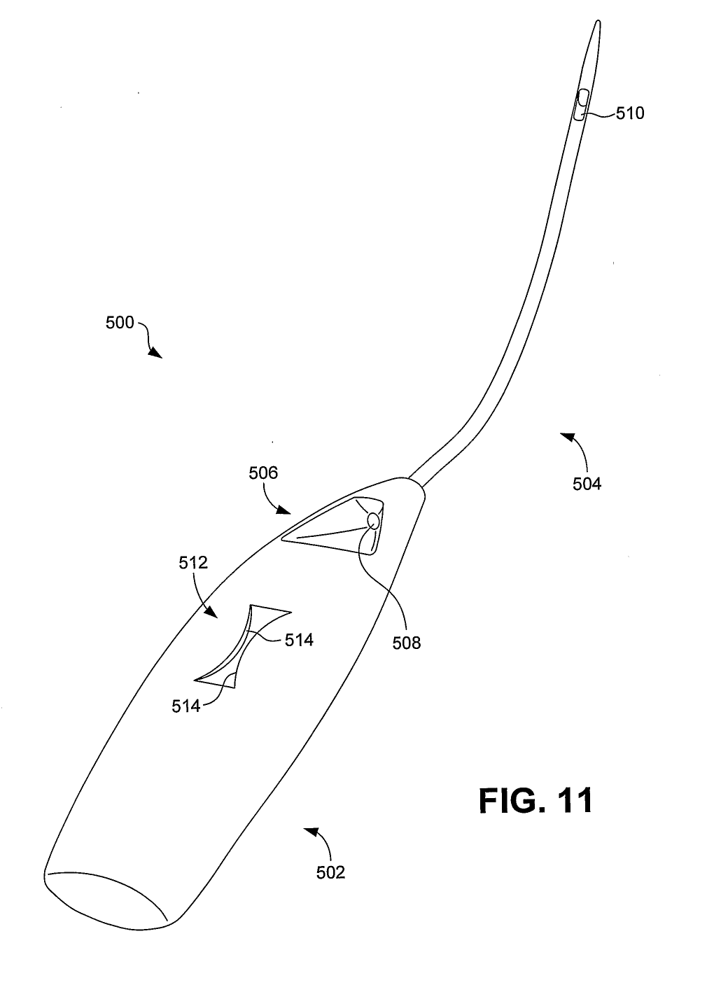

FIG. 11 illustrates a further embodiment of an introducer 500. As indicated in that figure, the introducer 500 comprises a handle 502 and a needle 504. As with the previously-described embodiments, the handle includes a port 506 that defines an orifice 508, which leads to an inner lumen of the needle 504. The needle 504 includes an opening 510 in communication with the inner lumen that enables a snare to be extended from the needle. Unlike the previously-described embodiments, however, the introducer 500 includes a cleat 512 comprising opposing inner surfaces 514 that are adapted to secure a snare relative to the introducer such that snare is positioned in a desired position along the inner lumen of the needle 504 when so secured.

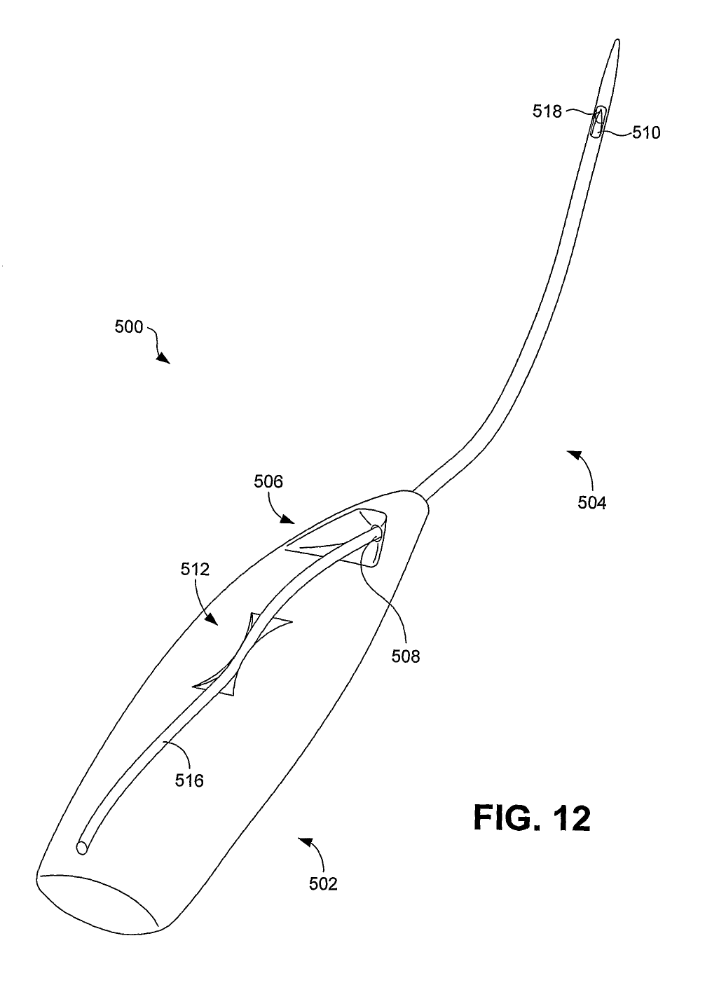

Turning to FIG. 12, securing of a snare 516 with the introducer 500 is depicted. As shown in that figure, the snare 516 is pushed into the cleat 512 such that the snare is securely clamped by the opposing inner surfaces 514 of the cleat. In some embodiments, the snare 516 can comprise indicia (not shown) that indicate what portion of the snare is to be secured within the cleat 512 such that indexing is provided as to important positions of the snare within the introducer 500. For example, indicia can be provided on the snare 516 at a position that, when aligned with the cleat, correspond to a position at which an implant coupling element 518 of the snare is positioned just within the opening 510 of the needle 504. In other embodiments, the snare 516 can comprise a complementary feature (not shown), such as mating indentations or protuberances, that are specifically adapted to interface with the cleat 512.

1. An introducer system comprising:
an introducer needle having a proximal end and a distal end and defining an inner lumen, the introducer needle further having an opening that provides access to the inner lumen;
and a snare having an implant coupling element, the snare being configured to be positioned within the inner lumen of the introducer needle, wherein the snare is extendable from the introducer needle opening to an extended position in which the implant coupling element is positioned outside of the inner lumen and retractable to a retracted position in which the implant coupling element is positioned within the inner lumen.

2. The system of claim 1, wherein the introducer needle opening is positioned adjacent the distal end of the introducer needle.

3. The system of claim 1, wherein the introducer needle distal end forms a tip configured to dissect tissue as the introducer needle is passed into the body through an external incision.

4. The system of claim 1, wherein the introducer needle is sized and shaped such that the distal end can be positioned at desired location within the body when the introducer needle is advanced through an external incision.

5. The system of claim 4, wherein the desired location is the vagina and the external incision is located in the paravaginal region or pararectal tissue.

6. The system of claim 1, wherein the introducer needle is elongated and curved.

7. The system of claim 1, wherein the implant coupling element comprises a loop configured to receive a portion of an implant.

8. The system of claim 7, wherein the loop is constructed of at least one of stainless steel and nitinol.

9. The system of claim 7, wherein the loop includes a constriction configured to securely clamp the implant.

10. The system of claim 7, wherein the snare comprises a tube and wherein the loop extends from the tube.

11. The system of claim 1, further comprising a handle that is secured to the proximal end of the introducer needle.

12. The system of claim 11, wherein the introducer needle comprises a second opening that provides access to the inner lumen.

13. The system of claim 12, wherein the handle comprises a port that provides access to the second opening of the introducer needle such that the snare can be positioned within the introducer needle inner lumen via the port and further removed from the introducer needle and handle via the port.

14. The system of claim 12, wherein the handle comprises a cleat configured to secure the snare in a predetermined position relative to the introducer needle.

15. The system of claim 14, wherein the predetermined position is the retracted position.

16. An introducer system comprising:
an elongated and curved introducer needle having a proximal end and a distal end, the introducer needle further having a first opening positioned adjacent the distal end and a second opening adjacent the proximal end and defining an inner lumen that extends between the first and second openings, the introducer needle further having a tip formed at the distal end, the tip being configured to dissect tissue as the introducer needle is passed into the body through an external incision;
a handle that is secured to the proximal end of the introducer needle, the handle having a port that provides access to the second opening of the introducer needle and the introducer needle inner lumen; and
an elongated snare having a proximal end and a distal end, the snare further having an implant coupling element positioned at its distal end and a grip element positioned at its proximal end, the snare being sized and shaped to be inserted into the introducer needle inner lumen via the handle port and introducer needle second opening, to be passed through the inner lumen to a retracted position in which the implant coupling element is adjacent the first opening but within the inner lumen, and to be extended to an extended position in which the implant coupling element is positioned outside of the inner lumen such that an implant can be coupled to the implant coupling element.

17. The system of claim 16, wherein the introducer needle is sized and shaped such that the distal end can be positioned within the vagina when the introducer needle has been advanced through the external pelvic incision.

18. The system of claim 16, wherein the introducer needle is sized and shaped such that the distal end can be positioned within the vaginal vault when the introducer needle has been advanced through a pararectal incision.

19. The system of claim 16, wherein the implant coupling element comprises a loop configured to receive a portion of an implant.

20. The system of claim 19, wherein the loop includes a constriction configured to securely clamp the implant.

21. The system of claim 16, wherein the handle comprises a cleat adjacent the port that is configured to secure the snare in a predetermined position relative to the introducer needle.

22-30. (canceled)

31. A method for positioning a mesh implant within the body, the method comprising:
passing a distal tip of an introducer needle through an external pelvic incision into the vagina, the introducer needle comprising an inner lumen;
extending an elongated snare from an opening adjacent the introducer needle distal tip to the vaginal introitus by pushing the snare through a port of a handle that is connected to the introducer needle, the port providing access to the introducer needle inner lumen;
connecting an arm of the mesh implant to the snare; and
retracting the snare back into the introducer needle by pulling the snare through the handle.
